(19) United States
(12) Patent Application Publication
(10) Pub. No.: US 2019/0216738 A1
(43) Pub. Date: Jul. 18, 2019

(54) PIRFENIDONE-CONTAINING TABLET AND CAPSULE FORMULATION

(71) Applicants: Laurus Labs Ltd, Hyderabad (IN); Alfred E. Tiefenbacher (GmbH & Co. KG), Hamburg (DE)

(72) Inventors: Chaitanya Yogananda Gujjar, Sangareddy District (IN); Susheel Prakash Uppala, Sangareddy District (IN); Harish Dhanala, Sangareddy District (IN); Nani Prasad Donga, Sangareddy District (IN); Srimannarayana Bandla, Sangareddy District (IN); Bala Ramesha Chary Rallabandi, Sangareddy District (IN); Siva Rama Krishna Velaga, Hyderabad (IN); Hendrik Schlehahn, Hamburg (DE)

(21) Appl. No.: 16/181,447

(22) Filed: Nov. 6, 2018

(30) Foreign Application Priority Data
Jan. 12, 2018 (IN) 201811001461

(51) Int. Cl. A61K 9/16; A61K 31/4418; A61K 9/48; A61K 9/20

(52) U.S. Cl. CPC A61K 9/1623; A61K 9/2095; A61K 9/4833; A61K 31/4418

(57) ABSTRACT

The present invention relates to granules containing pirfenidone and a sugar alcohol, and, furthermore, to the use of the granules for the preparation of an immediate-release tablet or capsule.

### CROSS-REFERENCE TO RELATED APPLICATIONS

This application is based on and claims the benefit of Indian Application No. 201811001461, filed on Jan. 12, 2018, entitled “Pirfenidone-containing Tablet and Capsule Formulation,” the content of which is incorporated by reference herein.

### BACKGROUND OF THE INVENTION

### Field of the Invention

The present invention relates to granules containing pirfenidone and a sugar alcohol, and, furthermore, to the use of the granules for the preparation of an immediate-release tablet or capsule.

### Description of Related Art

Pirfenidone is marketed under the tradename Esbrietx in the form of film-coated tablets, which contain 267, 534 or 801 mg pirfenidone, or as a hard capsule containing 267 mg pirfenidone, for the treatment of idiopathic pulmonary fibrosis (IPF). The recommended daily maintenance dosage of Esbriet® is 801 mg three times daily for a total of 2403 mg/day. Upon initiation of treatment, titration to the full dosage of 2403 mg/day over a 14-day period is required (days 1-7, 267 mg three times daily; days 8-14, 534 mg three times daily; days 15 onward, 801 mg three times daily).

The Esbriet® tablets and capsule provide for an immediate-release of the drug. The capsule contains, besides the drug, microcrystalline cellulose, croscarmellose sodium, povidone and magnesium stearate, while the tablet contains, in the core, the drug, microcrystalline cellulose, colloidal anhydrous silica, povidone K30, croscarmellose sodium and magnesium stearate. Pirfenidone is a crystalline powder and sparingly soluble in 1.0 N HCl, water and 1.0 N NaOH; the dissolution in water is pH independent (19 mg/ml at 25° C.). Pirfenidone has poor flowability characteristics, and in view of the fact that the Esbriet® capsule and tablets contain high concentrations of pirfenidone, in order to provide a tablet and capsule size that is suitable for oral administration, the state of the art focuses on improving the processability of the drug. The Esbriet® capsule and tablets are prepared by using wet-granulation.

EP-A-1 356 816 describes the preparation of a tablet containing 200 mg pirfenidone, in which the drug, lactose and a part of the contained carmellose calcium is subjected to wet-granulation using an aqueous solution of hydroxypropyl cellulose as a granulating fluid, followed by mixing the obtained granules with the remaining part of carmellose calcium and magnesium stearate, and subjecting the mixture to compression. Subsequently, the tablet core is coated with a film. This tablet is marketed in Japan under the tradename Pirespa® for the treatment of IPF.

It is stated in WO 2007/038315 that the Pirespa® tablet core contains about 70% pirfenidone and that a higher drug content would be desirable. WO 2007/038315 relates to a capsule formulation containing 70-95% pirfenidone and 5-30% pharmaceutical excipients. This application relates to the Esbriet® capsule, which may be prepared by milling a mixture of croscarmellose sodium, microcrystalline cellulose and pirfenidone, and subjecting the obtained mixture to wet-granulation using an aqueous povidone solution as granulating liquid. Subsequently, the granules are mixed with an additional amount of croscarmellose sodium and magnesium stearate, and, thereafter, the obtained mixture is filled into a capsule.

EP-A-2 735 306 discloses an extended-release tablet containing pirfenidone. The tablet is prepared by direct compression, in which pirfenidone and silicon dioxide are mixed in order to improve the flowability of the drug, followed by adding microcrystalline cellulose, low-viscosity hydroxypropyl methylcellulose (HPMC), high-viscosity HPMC and sodium stearyl fumarate, and subjecting the obtained mixture to compression. The low and high-viscosity HPMCs form the extended-release matrix, whereby the microcrystalline cellulose improves the tablet hardness.

WO 2017/172602 relates to the Esbriet® tablet. It is stated in the application that the particle size of pirfenidone can vary between the suppliers and that is was found that these particle size variations affect the hardness of the tablet. However, the particle size distribution of pirfenidone has no influence on the drug release characteristics, if the Dᵥ90 value is adjusted to 50-150 μm. Rather, the drug release characteristics depend on the solid fraction of the tablet (the ratio of the tablet's apparent density and true density): the higher the solid fraction, the longer the disintegration time.

The tablet is prepared by subjecting pirfenidone, a filler (preferably microcrystalline cellulose) and a glidant (preferably silica) to wet-granulation using an aqueous binder (preferably povidone) solution. The obtained granules are subsequently mixed with a disintegrant (preferably croscarmellose sodium), a lubricant (preferably magnesium stearate) and a glidant (preferably silica). Finally, the obtained mixture is subjected to compression, and the obtained tablet core is film-coated. The intragranular glidant is required to improve the flowability of pirfenidone.

### BRIEF SUMMARY OF THE INVENTION

One aspect of the present invention is to provide an immediate-release tablet or capsule for oral administration containing pirfenidone, which may contain the drug in a high concentration of at least 70% by weight and which can be prepared from a drug formulation that exhibits good flow properties. This aspect is attained by the subject matter as defined in the claims.

The present invention relates to granules containing pirfenidone and a sugar alcohol, and, furthermore, to the use of the granules for the preparation of an immediate-release tablet or capsule.

In one aspect of the invention, it was found that the particle size of pirfenidone affects the hardness of the tablet: the higher the particle size of pirfenidone, the lower the hardness of the tablet. It was further found that the desired hardness could not be achieved if the utilized pirfenidone has a particle size distribution Dᵥ90 of more than 400 μm. Thus, the particle size distribution of pirfenidone is preferably adjusted to Dᵥ90 =50-300 μm, preferably 100-200 μm and most preferred about 140-170 μm (determined by using Mastersizer 2000).

### DETAILED DESCRIPTION OF ILLUSTRATIVE EMBODIMENTS

The unit dosage form of the present invention is an immediate-release tablet or an immediate-release capsule for oral administration; preferably the tablet is a film-coated tablet. These unit dosage forms contain granules containing pirfenidone, a sugar alcohol, optionally a disintegrant and optionally a further pharmaceutical excipient, wherein the weight ratio of the sugar alcohol to pirfenidone is 2:100 to 30:100. Furthermore, these unit dosage forms may contain pirfenidone in a total amount of 267 mg, 534 mg or 801 mg. It was found that a glidant is not required in order to improve the flowability of pirfenidone, if the powder mixture to be subjected to granulation contains a sugar alcohol. Thus, in a preferred embodiment of the present invention, the granules do not contain a glidant.

It is preferred that the weight ratio of the sugar alcohol to pirfenidone is 3:100 to 25:100, wherein the granules to be filled into the capsule contain the sugar alcohol and pirfenidone preferably in a weight ratio of 5:100 to 15:100, more preferred 8:100 to 12:100 and most preferred is 10.5:100 (sugar alcohol:pirfenidone), and wherein the granules for tablets contain the sugar alcohol and pirfenidone more preferred in a weight ratio of 3.1:100 (sugar alcohol:pirfenidone).

The further pharmaceutical excipient, optionally contained in the granules of the present invention, may be selected from a binder and a filler. It is preferred that the granules contained in the tablet of the present invention are prepared by wet-granulation, in which case a binder is preferably contained in the granules. The preferred method for preparing the granules contained in the capsule of the present invention is dry-granulation, in which case no binder is required. It was found that it is difficult to fill granules prepared by wet-granulation into size “1” capsules, but larger capsules are less convenient to the patient upon swallowing. It is preferred that the granules of the present invention consist of pirfenidone, a disintegrant, a sugar alcohol, and optionally a binder and optionally a filler.

Typically, the sugar alcohol contained in the granules of the present invention is a C₄ to C₁₂ sugar alcohol, such as erythritol, xylitol, sorbitol, mannitol, maltitol, lactitol and isomalt. The preferred sugar alcohol contained in the granules of the tablet is mannitol, while the preferred sugar alcohol contained in the granules of the capsule is isomalt.

It is desired that the tablet core has a minimum hardness to remain intact through post-compaction processes, such as coating, transport/handling and packaging. The tablet core hardness for the 267-mg strength should be at least 60 N, and, for the 543-mg and 801-mg strengths, a hardness of at least 90 N and at least 120 N, respectively, is desired. It was found that a desired minimum tablet core hardness, and thus the tablet hardness, could not be achieved if a sugar alcohol is not present in the granules.

Examples of disintegrants include croscarmellose sodium, sodium starch glycolate, polyvinylpolypyrrolidone (crospovidone), carmellose sodium, carmellose potassium, polacrilin sodium, polacrilin potassium, silicified microcrystalline cellulose and low-substituted hydroxypropyl cellulose, whereby croscarmellose sodium is preferred.

The further pharmaceutical excipient contained in the tablet and capsule of the present invention may be selected from fillers, binders, glidants and lubricants.

Examples of fillers include microcrystalline cellulose, calcium hydrogen phosphate (anhydrous or dihydrate), lactose (anhydrous or monohydrate), dextrose, calcium carbonate, starch, pregelatinized starch, magnesium carbonate, silicified microcrystalline cellulose and powder cellulose, whereby microcrystalline cellulose is preferred.

Examples of binders include hydroxymethyl cellulose, hydroxypropyl cellulose, hydroxypropyl methylcellulose (Hypromellose), guar gum, maltodextrin, corn starch, polyvinylpyrrolidone (povidone) and vinylpyrrolidone/vinyl acetate copolymer (copovidone), wherein povidone is preferred.

Examples of glidants include silicon dioxide (silica), e.g. fumed (colloidal) silicon dioxide, talc and magnesium silicate, wherein silicon dioxide is preferred.

Examples of lubricants include magnesium stearate, calcium stearate, zinc stearate, stearic acid, sodium stearyl fumarate and glyceryl dibehenate, wherein the capsule preferably contains magnesium stearate and the tablet preferably contains sodium stearyl fumarate.

The capsule of the present invention is preferably prepared by a dry-granulation process comprising the steps:

(i) preparing a mixture containing pirfenidone, a sugar alcohol, optionally a first disintegrant and optionally a further first pharmaceutical excipient,

(ii) subjecting the mixture obtained in step (i) to compaction,

(iii) milling the compacted material obtained in step (ii) to obtain granules, wherein the weight ratio of the sugar alcohol to pirfenidone in the obtained granules is 2:100 to 30:100,

(iv) optionally mixing the granules with a second disintegrant and/or a further second pharmaceutical excipient, and

(v) filling the granules obtained in step (iii) or the mixture obtained in step (iv) into a capsule.

The dry-granulation process may be performed by roller compaction or slugging, wherein roller compaction is preferred. The further second pharmaceutical excipient is preferably a lubricant.

In a preferred embodiment of the process of the present invention, the capsule contains a mixture consisting of granules made from pirfenidone, a disintegrant and a sugar alcohol, and a lubricant, and the process comprises the steps:

(i) preparing a mixture consisting of pirfenidone, a disintegrant and a sugar alcohol,

(ii) subjecting the mixture obtained in step (i) to compaction,

(iii) milling the compacted material obtained in step (ii) to obtain granules,

(iv) mixing the granules obtained in step (iii) with a lubricant, and

(v) filling the mixture obtained in step (iv) into a capsule.

Preferably, the disintegrant is croscarmellose sodium, the sugar alcohol is isomalt and the lubricant is magnesium stearate.

The optionally film-coated tablet of the present invention, which contains pirfenidone, a sugar alcohol, optionally a disintegrant and optionally a further pharmaceutical excipient, is preferably prepared by wet-granulation comprising the steps:

(i) preparing a mixture containing pirfenidone, a sugar alcohol, optionally a first disintegrant and optionally a further first pharmaceutical excipient,

(ii) subjecting the mixture obtained in step (i) to wet granulation using an aqueous granulating liquid, wherein the weight ratio of the sugar alcohol to pirfenidone in the obtained granules is 2:100 to 30:100,

(iii) optionally mixing the granules obtained in step (ii) with a second disintegrant and/or a further second pharmaceutical excipient,

(iv) subjecting the granules obtained in step (ii) or the mixture obtained in step (iii) to compression to obtain the tablet.

It is preferred that the first further pharmaceutical excipient used in method step (i) is a filler, while the second pharmaceutical excipient used in method step (iii) may be selected from a filler, a glidant and a lubricant. It is preferred that, for the wet-granulation step (ii), an aqueous granulating liquid containing a binder, which is preferably a binder-containing aqueous solution, is used.

According to a preferred embodiment of the present invention, the tablet or the tablet core consists of a mixture consisting of granules made from pirfenidone, a first disintegrant, a sugar alcohol, a binder and optionally a first filler, a second disintegrant, a second filler, a glidant and a lubricant, wherein the process comprises the steps:

i) preparing a mixture consisting of pirfenidone, a first disintegrant, a sugar alcohol and optionally a first filler,

(ii) subjecting the mixture obtained in step (i) to wet granulation using an aqueous binder-containing solution as granulating liquid,

(iii) mixing the granules obtained in step (ii) with a second disintegrant, a second filler, a glidant and a lubricant,

(iv) subjecting the mixture obtained in step (iii) to compression to obtain the tablet.

The first and second disintegrant may be identical. In a preferred embodiment of the process of the present invention, the first and second disintegrant is croscarmellose sodium, the sugar alcohol is mannitol, the optionally contained first filler and the second filler are microcrystalline cellulose, the glidant is silicon dioxide and the lubricant is sodium stearyl fumarate.

The compressed tablets may optionally be coated with a film coating polymer. Examples of polymers for film coating are polyvinyl alcohol, hydroxypropyl methylcellulose, hydroxypropyl cellulose, whereby polyvinyl alcohol is preferred. The film coating layer may further comprise plasticizer and colorants. Preferably the film coating layer comprises polyvinyl alcohol, titanium dioxide, macrogol (PEG), talc and one or more colorants (e.g. iron oxide). Ready-made commercially available film coating systems containing polyvinyl alcohol polymers, like Opadry® Clear 06F590004, Opadry® Pink OY-S-34907, Opadry® II Purple 85F500082, Opadry® II Purple 85F500083, Opadry® Yellow 85F520311, Opadry® II Brown 85F565071 and Opadry® Orange 85F530178, can be used.

It was found that the particle size of pirfenidone affects the hardness of the tablet: the higher the particle size of pirfenidone, the lower the hardness of the tablet. It was found that the desired hardness could not be achieved if the utilized pirfenidone has a particle size distribution Dᵥ90 of more than 400 μm. Thus, the particle size distribution of pirfenidone is preferably adjusted to Dᵥ90 =50-300 μm, preferably 100-200 μm and most preferred about 140-170 μm (determined by using Mastersizer 2000).

The optionally film-coated tablet and capsule of the present invention preferably contains pirfenidone in an amount of at least 70% by weight. Preferably the capsule of the present invention comprises 80-90% by weight of pirfenidone, 2-10% by weight of a disintegrant, 5-15% by weight of a sugar alcohol, 0-10% by weight of a binder and 0.1-5% by weight of a lubricant, for example, 84.8% by weight of pirfenidone, 6.0% by weight of a disintegrant (e.g. croscamellose sodium), 8.9% by weight of a sugar alcohol (e.g. isomalt) and 0.3% by weight of a lubricant (e.g. magnesium stearate). Preferably the tablet core of the present invention comprises 85-90% by weight of pirfenidone, 1-10% by weight of a disintegrant, 1-10% by weight of a sugar alcohol, 0.5-7% by weight of a binder, 1-10% by weight of a filler, 0.1-5% by weight of a lubricant and 0.1-5% by weight of a glidant. More preferred the tablet core of the present invention comprises 85-90% by weight of pirfenidone, 1-5% by weight of a disintegrant, 1-5% by weight of a sugar alcohol, 0.5-5% by weight of a binder, 1-8% by weight of a filler, 0.1-3% by weight of a lubricant and 0.1-3% by weight of a glidant, for example, 87% by weight of pirfenidone, 2.2% by weight of a disintegrant (e.g. croscarmellose sodium) and 3.2% by weight of a sugar alcohol (e.g. mannitol) or 2.7% by weight of disintegrant (e.g. croscarmellose sodium) and 2.7% by weight of sugar alcohol (e.g. mannitol), 2.2% by weight of a binder (e.g. povidone), 4.5% by weight of a filler (e.g. microcrystalline cellulose), 0.6% by weight of a lubricant (e.g. sodium stearyl fumarate) and 0.3% by weight of a glidant (e.g. colloidal silicon dioxide). Preferably the capsule of the present invention comprises 267 mg of pirfenidone, whereas the tablet core of the present invention preferably comprises pirfenidone in a total amount of 267 mg, 534 mg or 801 mg.

The following examples are intended to further illustrate the present invention.

### EXAMPLES

In the examples, crystalline pirfenidone having a particle size distribution of Dᵥ90 =100-200 μm was used. Particle size distribution was determined by using laser diffraction with a Mastersizer 2000 (by Malvern Intr. Ltd.) in dry dispersion mode. Dᵥ90 corresponds to the particle size at 90% of the cumulative volume distribution.

The dissolution tests were performed according to general chapter (711) DISSOLUTION of USP40-NF35 and the US FDA recommended dissolution method for pirfenidone capsule: apparatus II (paddle; with sinker in case of capsules), speed: 50 rpm, medium: deionized water, volume: 1000 ml, sampling times: 5, 10, 15, 20, 30, and 45 min as well as optionally 60 min.

Bulk density and tapped density were determined according to general chapter (616) BULK DENSITY AND TAPPED DENSITY OF POWDERS of USP40-NF35. Hardness was determined according to European Pharmacopeia 9.2, 2.9.8.

Disintegration time was determined according to general chapter (701) DISINTEGRATION of USP40-NF35.

Friability was determined according to general chapter (1216) TABLET FRIABILITY of USP40-NF35.

Compressibility Index and Hausner's Ratio were calculated from tapped and bulk density (Compressibility Index=(tapped density−bulk density)/tapped density*100; Hausner's Ratio=tapped density/bulk density).

### Examples 1-4

### Capsules

TABLE 1

| Ingredients | Ex. 1 | Ex. 2 | Ex. 3 | Ex. 4 |
|---|---|---|---|---|
| Stage-A (Blending & Compaction) | mg/cap | | | |
| Pirfenidone | 267.267 | 267.267 | 267.267 | 267.000 |
| Croscarmellose Sodium | 28.000 | 16.000 | 22.000 | 19.000 |
| (Ac-Di-Sol ®) | | | | |
| Isomalt (Galen IQ 721) | 27.733 | 27.733 | 27.733 | 28.000 |
| Stage- B (Lubrication) | | | | |
| Magnesium Stearate | 1.000 | 1.000 | 1.000 | 1.000 |
| (Ligamed ® MF-2-V) | | | | |
| Fill weight (mg) | 324.000 | 312.000 | 318.000 | 315.000 |
| Stage-C (Encapsulation) | | | | |
| Hard Gelatin Capsule | 75.000 | 75.000 | 75.000 | 75.000 |
| Shells (Size“1”) | | | | |
| Capsule weight (mg) | 399.000 | 387.000 | 393.000 | 390.000 |
| Physical Parameters (Blend) | | | | |
| Dᵥ90 of pirfenidone drug substance | 145 | 145 | 145 | 145 |
| Bulk Density (g/ml) | 0.556 | 0.573 | 0.548 | 0.553 |
| Tapped Density (g/ml) | 0.798 | 0.796 | 0.807 | 0.798 |
| Compressibility Index (%) | 30.357 | 28.000 | 32.099 | 30.702 |
| Hausner's Ratio | 1.436 | 1.389 | 1.473 | 1.443 |
| Physical Parameters (Capsules) | | | | |
| Average Lock Length (mm) | 18.98 | 18.95 | 19.01 | 19.00 |
| Average Weight (mg) | 399.8 | 386.5 | 393.7 | 390.6 |
| Disintegration time (min) | 2-3 min | 2-3 min | 2-3 min | 2-3 min |

Procedure:

The dispensed quantities of stage A materials are sifted through a suitable sieve (e.g. #30 mesh) and blended for a suitable time (e.g. 5 minutes) in a blender. The sifted materials are compacted using roller compactor with suitable set of parameters. The compacts are milled to the required sized granules (e.g. approx. 700 μm). Milled granules were sifted through a suitable sieve (e.g. #25 mesh) and the sized granules are blended with extra granular materials of stage B, in blender for a suitable time (for example 5 minutes). The lubricated granules are filled into size 1 hard gelatin capsules.

TABLE 2

| | Mean Cumulative % Labelled Amount Dissolved [%] | | | | | | |
|---|---|---|---|---|---|---|---|
| Example | 5 min | 10 min | 15 min | 20 min | 30 min | 45 min | 60 min |
| Esbriet ® 267 mg capsules | 58 | 78 | 84 | 90 | 98 | 102 | 103 |
| (Lot no. 3056759) | | | | | | | |
| Example 1 | 64 | 81 | 91 | 96 | 100 | 101 | 101 |
| Example 2 | 56 | 75 | 87 | 93 | 99 | 101 | 102 |
| Example 3 | 60 | 78 | 88 | 93 | 99 | 101 | 101 |
| Example 4 | 50 | 66 | 76 | 84 | 93 | 98 | 99 |

### Examples 5-7

### Tablets

TABLE 3

| Ingredients | Ex. 5 | Ex. 6 | Ex. 7 |
|---|---|---|---|
| Stage-A (Sifting & Blending) | mg/tab | | |
| Pirfenidone | 810.388 | 803.007 | 803.007 |
| Mannitol (Pearlitol ® SD200) | 195.612 | 115.993 | 104.993 |
| Sodium Starch Glycolate (Explotab ®) | 15.000 | — | — |
| Stage-B (Binder Solution) | | | |
| Hypromellose | 18.000 | 24.000 | 28.000 |
| (Methocel ® E15 Premium LV) | | | |
| Water, Purified | q.s. | q.s. | q.s. |
| Stage-C (Blending & Lubrication) | | | |
| Sodium Starch Glycolate (Explotab ®) | 9.000 | 20.000 | 16.000 |
| Silica Colloidal Anhydrous (Aerosil ® | 6.000 | 6.000 | 6.000 |
| 200) | | | |
| Sodium Stearyl Fumarate | — | 6.000 | 6.000 |
| Magnesium Stearate | 6.000 | — | — |
| Core Tablet Weight (mg) | 1060.000 | 975.000 | 964.000 |
| Stage-D (Coating) | | | |
| Opadry ® Clear 06F590004 | 30.000 | 25.000 | 25.000 |
| Water, Purified | q.s. | q.s. | q.s. |
| Coated Tablet Weight (mg) | 1090.000 | 1000.000 | 989.000 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet mass was dried in Fluidized bed drier. The dried granules were milled. The sized granules were sifted through a suitable sieve (e.g. #25 mesh) and blended with sifted extra granular materials of Stage-C. The resulting mixture was compressed to (core) tablets in a tabletting machine and film-coated (using stage-D film coating) in an automated coating machine with perforated pan.

TABLE 4

| Blend Parameters | Ex. 5 | Ex. 6 | Ex. 7 |
|---|---|---|---|
| Bulk Density (g/ml) | 0.468 | 0.451 | 0.490 |
| Tapped Density (g/ml) | 0.570 | 0.552 | 0.610 |
| Compressibility Index (%) | 17.895 | 18.29 | 21.428 |
| Hausner's Ratio | 1.218 | 1.223 | 1.273 |

TABLE 5

| | Ex. 5 | Ex. 6 | Ex. 7 |
|---|---|---|---|
| Tablet Core Parameters | | | |
| Tablet Weight (mg) | 1060-1064 | 965-975 | 960-970 |
| Hardness (Newtons) | 150-156 | 145-155 | 140-150 |
| Thickness (mm) | 6.39-6.41 | 6.00-6.10 | 6.60-6.70 |
| Disintegration Time (min) | 2 min 0 sec | 2 min 05 sec | 3 min 40 sec |
| Friability (%) | 0.03 | 0.061 | 0 |
| Coated Tablet Parameters | | | |
| Tablet Weight (mg) | 980-990 | 980-990 | 980-990 |
| Thickness (mm) | 6.80-6.90 | 6.70-6.80 | 6.80-6.90 |
| Disintegration Time (min) | 7 min 38 sec | 8-9 min | 7 min 38 sec |

TABLE 6

| | Mean Cumulative % Labelled Amount Dissolved [%] | | | | | | |
|---|---|---|---|---|---|---|---|
| | | | | 20 | | | |
| Example | 5 min | 10 min | 15 min | min | 30 min | 45 min | 60 min |
| Example 5 | 59 | 91 | 96 | 99 | 103 | 105 | 105 |
| Example 6 | 59 | 86 | 92 | 95 | 96 | 97 | 97 |
| Example 7 | 44 | 75 | 90 | 95 | 99 | 101 | 102 |

### Examples 8 and 9

### Tablets

TABLE 7

| | | |
|---|---|---|
| Ingredients | Ex. 8 | Ex. 9 |

| | |
|---|---|
| Stage-A (Dispensing & Blending) | mg/tab |

| | | |
|---|---|---|
| Pirfenidone | 802.605 | 803.893 |
| Mannitol (Pearlitol ® 200 SD) | 106.395 | 105.107 |
| Sodium Starch Glycolate (Explotab ®) | — | 8.000 |
| Stage-B (Binder Solution) | | |
| Povidone (Plasdone ® K-29/32) | 25.000 | 25.000 |
| Water, Purified | q.s. | q.s. |
| Stage-C (Blending & Lubrication) | | |
| Sodium Starch Glycolate (Explotab ®) | 16.000 | 8.000 |
| Silica, Colloidal Anhydrous | 6.000 | 6.000 |
| (Aerosil ® 200 Pharma) | | |
| Sodium Stearyl Fumarate | 6.000 | 6.000 |
| Core tablet weight (mg) | 962.000 | 962.000 |
| Stage-D (Coating) | | |
| Opadry ® Pink OY-S-34907 | 25.000 | 25.000 |
| Water, Purified | q.s. | q.s. |
| Coated Tablet weight (mg) | 987.000 | 987.000 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet granules were dried. The dried granules were milled. The sized granules were sifted through a suitable sieve (e.g. #25 mesh) and blended with sifted extra granular materials of Stage-C. The resulting mixture was compressed to (core) tablets in a tabletting machine and film-coated (using stage-D film coating) in an automated coating machine with perforated pan.

TABLE 8

| Blend Parameters | Ex. 8 | Ex. 9 |
|---|---|---|
| Bulk Density (g/ml) | 0.526 | 0.490 |
| Tapped Density (g/ml) | 0.623 | 0.610 |
| Compressibility Index (%) | 16 | 21.428 |
| Hausner's Ratio | 1.18 | 1.273 |

TABLE 9

| | Ex. 8 | Ex. 9 |
|---|---|---|
| Tablet Core Parameters | | |
| Tablet Weight (mg) | 960-970 | 960-970 |
| Hardness (Newtons) | 140-155 | 160-175 |
| Thickness (mm) | — | 6.55-6.65 |
| Disintegration Time (min) | 2 min 30 sec | 3-4 min |
| Friability (%) | 0 | 0 |
| Coated Tablet Parameters | | |
| Tablet Weight (mg) | 980-990 | 980-990 |
| Thickness (mm) | — | 6.70-6.80 |
| Disintegration Time (min) | 5-6 min | 8-9 min |

TABLE 10

| | Mean Cumulative % Labelled Amount Dissolved [%] | | | | | | |
|---|---|---|---|---|---|---|---|
| | | | | 20 | | | |
| Example | 5 min | 10 min | 15 min | min | 30 min | 45 min | 60 min |
| Example 8 | 72 | 97 | 102 | 103 | 103 | 103 | 103 |
| Example 9 | 33 | 83 | 98 | 100 | 100 | 102 | 101 |

### Examples 10 and 11

### Tablets

TABLE 11

| | | |
|---|---|---|
| Ingredients | Ex. 10 | Ex. 11 |

| | |
|---|---|
| Stage-A (Sifting & Blending) | mg/tab |

| | | |
|---|---|---|
| Pirfenidone | 803.893 | 801.802 |
| Mannitol | 50.000 | 30.000 |
| (Pearlitol ® SD 200) | | |
| Microcrystalline Cellulose | 51.107 | — |
| (Pharmacel ® 101) | | |
| Croscarmellose Sodium | 10.000 | 5.000 |
| (Ac-di-sol ®) | | |
| Stage-B (Binder Solution) | | |
| Povidone (Plasdone ® K-29/32) | 35.000 | 20.000 |
| Water, Purified | q.s. | q.s. |
| Stage-C (Blending & Lubrication) | | |
| Microcrystalline Cellulose | — | 40.198 |
| (Pharmacel ® 101) | | |
| Croscarmellose Sodium | 10.000 | 15.000 |
| (Ac-di-sol ®) | | |
| Silica, Colloidal Anhydrous | 3.000 | 3.000 |
| (Aerosil ® 200 Pharma) | | |
| Sodium Stearyl Fumarate | 6.000 | 6.000 |
| Core Tablet Weight (mg) | 969.000 | 921.000 |
| Stage-D (Coating) | | |
| Opadry ® II Purple 85F500083 | 27.000 | 24.000 |
| Water, Purified | q.s. | q.s. |
| Coated Tablet Weight (mg) | 996.000 | 945.000 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet mass was milled and the sized granules were dried. The dried granules were sifted through a suitable sieve (e.g. #20 mesh) and blended with sifted extra granular materials of Stage-C. The resulting mixture was compressed to (core) tablets in a tabletting machine and film-coated (using stage-D film coating) in an automated coating machine with perforated pan.

TABLE 12

| Blend Parameters | Ex. 10 | Ex. 11 |
|---|---|---|
| Bulk Density (g/ml) | 0.440 | 0.449 |
| Tapped Density (g/ml) | 0.551 | 0.592 |
| Compressibility Index (%) | 20.19 | 24.15 |
| Hausner's Ratio | 1.252 | 1.318 |

TABLE 13

| | Ex. 10 | Ex. 11 |
|---|---|---|
| Tablet Core Parameters | | |
| Tablet Weight (mg) | 966-977 | 917-924 |
| Hardness (Newtons) | 166-175 | 137-156 |
| Thickness (mm) | 6.69-6.72 | 6.95-7.02 |
| Disintegration Time (min) | 3 min | 0 min 36 sec |
| Friability (%) | 0.09 | 0 |
| Coated Tablet Parameters | | |
| Tablet Weight (mg) | 995-1000 | 940-950 |
| Thickness (mm) | 6.77-6.79 | 7.00-7.05 |
| Disintegration Time (min) | 4-5 min | 1 min |

TABLE 14

| | Mean Cumulative % Labelled Amount Dissolved [%] | | | | | | |
|---|---|---|---|---|---|---|---|
| | | 10 | | | | 45 | |
| Example | 5 min | min | 15 min | 20 min | 30 min | min | 60 min |
| Example 10 | 53 | 94 | 97 | 98 | 99 | 99 | 99 |
| Example 11 | 93 | 99 | 100 | 101 | 102 | 102 | 103 |

### Examples 12-15

### Tablets

TABLE 15

| | Ex. 12 | Ex. 13 | Ex. 14 | Ex. 15 |
|---|---|---|---|---|
| Ingredients | 267 mg | 267 mg | 534 mg | 801 mg |
| Stage-A (Sifting) | mg/tab | | | |
| Pirfenidone | 267.000 | 267.964 | 534.000 | 801.000 |
| Mannitol (Pearlitol ® SD | 10.000 | 34.369 | 20.000 | 30.000 |
| 200) | | | | |
| Croscarmellose Sodium | 1.667 | — | 3.334 | 5.000 |
| Sodium Starch Glycolate | — | 2.667 | — | — |
| (Primogel ®) | | | | |
| Stage-B (Binder Solution) | | | | |
| Povidone (Plasdone ® K- | 6.667 | 8.333 | 13.334 | 20.000 |
| 29/32) | | | | |
| Water, Purified | q.s. | q.s. | q.s. | q.s. |
| Stage-C (Blending & | | | | |
| Lubrication) | | | | |
| Microcrystalline Cellulose | 13.666 | — | 27.332 | 41.000 |
| (Comprecel/Pharmacel ® PH | | | | |
| 101) | | | | |
| Sodium Starch Glycolate | — | 2.667 | — | — |
| (Primogel ®) | | | | |
| Croscarmellose Sodium | 5.000 | — | 10.000 | 15.000 |
| Silica, Colloidal Anhydrous | 1.000 | 2.000 | 2.000 | 3.000 |
| Sodium Stearyl Fumarate | 2.000 | 2.000 | 4.000 | 6.000 |
| Core Tablet Weight (mg) | 307.000 | 320.000 | 614.000 | 921.000 |
| Stage-D (Coating) | | | | |
| Opadry ® Yellow 85F520311 | 8.000 | — | — | — |
| Opadry ® II Brown | — | 8.000 | — | — |
| 85F565071 | | | | |
| Opadry ® Orange 85F530178 | — | — | 16.000 | — |
| Opadry ® II Purple | — | — | — | 24.000 |
| 85F500083 | | | | |
| Water, Purified | q.s. | q.s. | q.s. | q.s. |
| Coated Tablet Weight (mg) | 315.000 | 328.000 | 630.000 | 945.000 |

Procedure:

The stage-A materials were sifted through a suitable sieve (e.g. # 30 mesh), loaded into a rapid mixer granulator and mixed for a suitable time (e.g. 5 minutes). The binder solution was prepared by dispersing binder of stage-B into the purified water under stirring. The binder solution was slowly added into the rapid mixer granulator to obtain a wet mass. The wet mass was milled through a 6350 μm screen and the wet granules were dried. The dried granules were sifted through a suitable sieve (e.g. # 20 mesh) and retains collected were milled through a 1016 μm screen. The sized granules blended with extra granular materials of Stage-C (sifted in advance through a suitable sieve, e.g. # 30 mesh). The resulting mixture was compressed to (core) tablets in a tabletting machine. The coating solution was prepared by dispersing the coating material (stage-D) directly in to the purified water under stirring (20% w/w solids) and stirring was continued for a suitable time (e.g. 45 minute). The (core) tablets were coated with the coating solution by using an automated coating machine with perforated pan.

TABLE 16

| Blend Parameters | Ex. 12 | Ex. 13 | Ex. 14 | Ex. 15 |
|---|---|---|---|---|
| Dᵥ90 of pirfenidone drug | 145 | 150 | 145 | 145 |
| substance | | | | |
| Bulk Density (g/ml) | 0.449 | 0.497 | 0.449 | 0.424 |
| Tapped Density (g/ml) | 0.592 | 0.644 | 0.592 | 0.538 |
| Compressibility Index (%) | 24.15 | 22.82 | 24.15 | 21.212 |
| Hausner's Ratio | 1.318 | 1.295 | 1.318 | 1.269 |

TABLE 17

| | Ex. 12 | Ex. 13 | Ex. 14 | Ex. 15 |
|---|---|---|---|---|
| Tablet Core Parameters | | | | |
| Tablet Weight (mg) | 307 | 320 | 614.5 | 923.5 |
| Hardness (Newtons) | 90 | 95-105 | 110 | 166 |
| Thickness (mm) | 5.30 | 5.06 | 6.82 | 6.93 |
| Disintegration Time (min) | 30 sec | 2-3 | 34 sec | 2 |
| Friability (%) | 0.09 | 0 | 0 | 0 |
| Coated Tablet Parameters | | | | |
| Tablet Weight (mg) | 315 | 325-331 | 630.5 | 947.5 |
| Thickness (mm) | 5.35 | 5.13 | 6.83 | 7.00 |
| Disintegration Time (min) | 1 | 5-6 | 1 | 3 |

TABLE 18

| | Mean Cumulative % Labelled Amount Dissolved [%] | | | | | | |
|---|---|---|---|---|---|---|---|
| | | 10 | | | | 45 | |
| Example | 5 min | min | 15 min | 20 min | 30 min | min | 60 min |
| Example 12 | 90 | 97 | 101 | 102 | 102 | 103 | 103 |
| Example 13 | 56 | 92 | 98 | 100 | 101 | 101 | 101 |
| Example 14 | 93 | 97 | 100 | 101 | 101 | 102 | 102 |
| Example 15 | 81 | 91 | 93 | 95 | 97 | 99 | 100 |

### Examples 16-18

### Tablets

TABLE 19

| Ingredients | Ex. 16 | Ex. 17 | Ex. 18 |
|---|---|---|---|
| Stage-A (Sifting) | mg/tab | | |
| Pirfenidone | 267.454* | 534.909* | 802.364* |
| Mannitol (Pearlitol ® 200 SD) | 8.333 | 16.667 | 25.000 |
| Croscarmellose Sodium | 3.333 | 6.667 | 10.000 |
| (Ac-Di-Sol ® SD-711) | | | |
| Stage-B (Binder Solution) | | | |
| Povidone (Plasdone ™ K-29/32) | 6.667 | 13.334 | 20.000 |
| Water, Purified | q.s. | q.s. | q.s. |
| Stage-C (Blending & Lubrication) | | | |
| Microcrystalline Cellulose | 13.212* | 26.424* | 39.636* |
| (Pharmacel ® 101) | | | |
| Croscarmellose Sodium | 5.000 | 10.000 | 15.000 |
| (Ac-Di-Sol ® SD-711) | | | |
| Colloidal Silicon Dioxide (Aerosil ® | 1.000 | 2.000 | 3.000 |
| 200 Pharma) | | | |
| Sodium Stearyl Fumarate | 2.000 | 4.000 | 6.000 |
| Core Tablet Weight (mg) | 307.000 | 614.000 | 921.000 |
| Stage-D (Coating) | | | |
| Opadry ® II Yellow 85F520311 | 8.000 | — | — |
| Opadry ® II Orange 85F530178 | — | 16.000 | — |
| Opadry ® II Purple 85F500083 | — | — | 24.000 |
| Water, Purified | q.s. | q.s. | q.s. |
| Coated Tablet Weight (mg) | 315.000 | 630.000 | 945.000 |

*Adiusted for drug assay and water content (Theoretical weight of pirfenidone = 267.000 mg, 534.000 mg and 801.000 mg and of microcrystalline cellulose = 13.667 mg, 27.332 mg and 41.000 mg).

Procedure:

The stage-A materials were sifted through a suitable sieve (e.g. # 30 mesh), loaded into a rapid mixer granulator and mixed for a suitable time (e.g. 5 minutes). The binder solution was prepared by dispersing binder of stage-B into the purified water under stirring. The binder solution was slowly added into the rapid mixer granulator to obtain a wet mass. The wet mass was milled through a 6350 μm screen and the wet granules were dried. The dried granules were sifted through a suitable sieve (e.g. # 20 mesh) and retains collected were milled through a 1016 μm screen. The sized granules blended with extra granular materials of Stage-C (sifted in advance through a suitable sieve, e.g. # 30 mesh). The resulting mixture was compressed to (core) tablets in a tabletting machine. The coating solution was prepared by dispersing the coating material (stage-D) directly in to the purified water under stirring (20% w/w solids) and stirring was continued for a suitable time (e.g. 45 minute). The (core) tablets were coated with the coating solution by using an automated coating machine with perforated pan.

TABLE 20

| Blend Parameters | Ex. 16 | Ex. 17 | Ex. 18 |
|---|---|---|---|
| Dᵥ90 of pirfenidone drug substance | 145 μm | 145 μm | 145 μm |
| Bulk Density (g/ml) | 0.433 | 0.406 | 0.433 |
| Tapped Density (g/ml) | 0.552 | 0.522 | 0.552 |
| Compressibility Index (%) | 21.67 | 22.22 | 21.67 |
| Hausner's Ratio | 1.276 | 1.286 | 1.276 |

TABLE 21

| | Ex. 16 | Ex. 17 | | Ex. 18 | |
|---|---|---|---|---|---|
| Tablet Core Parameters | | | | | |
| Average Tablet Weight (mg) | 307.6 | 614.4 | | 921.5 | |
| Hardness (Newtons) | 82-92 | 112-115 | | 138-152 | |
| Thickness (mm) | 5.18-5.25 | 6.69-6.72 | | 6.98-7.02 | |
| Disintegration Time | 36 sec | 42 | sec | 50 | sec |
| Friability (%) | 0 | 0.03 | | 0 | |
| Coated Tablet Parameters | | | | | |
| Average Tablet Weight (mg) | 314.8 | 628.6 | | 947 | |
| Thickness (mm) | 5.27-5.29 | 6.80-6.83 | | 7.03-7.09 | |
| Disintegration Time (min) | 1 min 20 sec | 1 | min | 1.5 | min |

TABLE 22

| | Mean Cumulative % Labelled Amount Dissolved | | | | | |
|---|---|---|---|---|---|---|
| | [%] | | | | | |
| | | | 15 | | | |
| Example | 5 min | 10 min | min | 20 min | 30 min | 45 min |
| Esbriet ® | 73 | 94 | 98 | 100 | 101 | 100 |
| tablets 267 mg | | | | | | |
| (Lot no. M0009M7) | | | | | | |
| Example 16 | 85 | 91 | 96 | 98 | 99 | 99 |
| Example 17 | 92 | 95 | 97 | 99 | 99 | 100 |
| Esbriet ® | 33 | 86 | 93 | 96 | 98 | 101 |
| tablets 801 mg | | | | | | |
| (Lot no. M1000M1) | | | | | | |
| Example 18 | 80 | 99 | 101 | 102 | 102 | 102 |

### Comparative Example 1

### Capsule

TABLE 23

| Ingredients | Comp. Ex. 1 |
|---|---|
| Stage - A (Dry mix) | mg/tab |
| Pirfenidone | 267.481 |
| Microcrystalline Cellulose (Avicel ® PH 102) | 23.519 |
| Croscarmellose Sodium (Ac-Di-Sol ® SD 711) | 26.500 |
| Stage - B (Granulation) | |
| Povidone (Plasdone ® K 29/32) | 6.000 |
| Water, Purified | q.s. |
| Stage - C (Blending & Lubrication) | |
| Magnesium Stearate | 1.500 |
| Fill weight (mg) | 325.000 |
| Stage - D (Encapsulation) | |
| Hard Gelatin Capsule Shells (Size “1”) | 96.000 |
| Capsule weight (mg) | 421.000 |
| Physical Parameters (granules) | |
| Bulk Density (g/ml) | 0.422 |
| Tapped Density (g/ml) | 0.552 |
| Compressibility Index (%) | 23.040 |
| Hausner's Ratio | 1.305 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet granules were dried. The dried granules were milled. The sized granules were sifted through a suitable sieve (e.g. #30 mesh) and blended with sifted extra granular materials of Stage-C for a suitable time. The lubricated blend could not be filled into size “1” capsules. Thus, the dry-granulation process is preferred.

### Comparative Examples 2-4

### Tablets

TABLE 24

| | Comp. | Comp. | Comp. |
|---|---|---|---|
| Ingredients | Ex. 2 | Ex. 3 | Ex. 4 |
| Stage - A (Blending) | mg/tab | | |
| Pirfenidone | 268.018 | 268.018 | 268.018 |
| Mannitol (Pearlitol ® SD 200) | 42.982 | 64.982 | — |
| Dicalcium Phosphate Dihydrate | — | — | 67.982 |
| Hypromellose | 6.000 | — | 6.000 |
| (Methocel ® E5 Premium LV) | | | |
| Xanthan Gum | — | 7.000 | — |
| Sodium Starch Glycolate | 6.000 | 8.000 | 6.000 |
| (Explotab ®) | | | |
| Sodium Stearyl Fumarate | 2.000 | 2.000 | — |
| Magnesium Stearate | — | — | 2.000 |
| Core tablet weight (mg) | 325.000 | 350.000 | 350.000 |

Procedure:

The dispensed quantities were sifted through suitable sieve (e.g. #30 mesh). The sifted materials were blended for a suitable time in a blender. The resulting mixture was compressed to core tablets on a tabletting machine.

The desired hardness (above 60 N) could not be achieved. The direct compression process failed to give the desired hardness for the tablets.

### Comparative Examples 5 and 6

### Tablets

TABLE 25

| | Comp. | Comp. |
|---|---|---|
| Ingredients | Ex. 5 | Ex. 6 |
| Stage - A (Sifting & Blending) | mg/tab | |
| Pirfenidone | 268.018 | 268.018 |
| Dicalcium Phosphate Dihydrate (DICOFOS ® D014) | 67.982 | 67.982 |
| Sodium Starch Glycolate | 5.000 | 5.000 |
| Stage-B (Binder Solution) | | |
| Hypromellose (Methocel ® E5 Premium LV) | 6.000 | 6.000 |
| Water, Purified | q.s. | q.s. |
| Stage-C (Blending & Lubrication) | | |
| Sodium Starch Glycolate | 3.000 | 3.000 |
| Silica, Colloidal Anhydrous (Aerosil ® 200 Pharma) | — | 1.800 |
| Magnesium Stearate | 2.000 | 2.000 |
| Core tablet weight (mg) | 352.000 | 353.800 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet mass was dried in fluidized bed drier. The dried granules were sifted through a suitable sieve (e.g. #25 mesh) and retains were milled. The sized granules were blended with sifted extra granular materials of Stage-C. The resulting mixture was compressed to core tablets in a tabletting machine.

The desired hardness (above 60 N) could not be achieved without a sugar alcohol present in the granules. Maximum hardness achieved for both the batches was 45 N.

### Comparative Examples 7-8

### Tablets

TABLE 26

| Ingredients | Comp. Ex. 7 | Comp. Ex. 8 |
|---|---|---|
| Stage-A (Dispensing & Blending) | mg/tab | |
| Pirfenidone | 803.007 | 803.893 |
| Microcrystalline Cellulose | 104.993 | 103.107 |
| (Comprecel ® M 102 D) | | |
| Croscarmellose Sodium (Ac-Di-sol ®) | — | 8.000 |
| Stage-B (Binder solution) | | |
| Povidone (Plasdone ® K-29/32) | 28.000 | 35.000 |
| Water, Purified | q.s. | q.s. |
| Stage-C (Blending & Lubrication) | | |
| Croscarmellose Sodium (Ac-Di-sol ®) | 16.000 | 8.000 |
| Silica, Colloidal Anhydrous | 6.000 | 6.000 |
| (Aerosil ® 200 Pharma) | | |
| Sodium Stearyl Fumarate | 6.000 | 6.000 |
| Core tablet weight (mg) | 964.000 | 970.000 |

Procedure:

The stage-A materials were sifted through a suitable sieve (e.g. #30 mesh), loaded into a rapid mixer granulator and mixed for a suitable time. The binder solution was prepared by dispersing binder of stage-B into the purified water. The binder solution was slowly added into the rapid mixer granulator to obtain a wet mass. The wet mass was dried in fluidized bed drier. The dried granules were sifted through a suitable sieve (e.g. #25 mesh) and retains were milled. The sized granules were blended with sifted extra granular materials of Stage-C. The resulting mixture was compressed to core tablets in a tabletting machine.

TABLE 27

| | Comp. Ex. 7 | Comp. Ex. 8 |
|---|---|---|
| Blend Parameters | | |
| Bulk Density (g/ml) | 0.411 | 0.414 |
| Tapped Density (g/ml) | 0.532 | 0.506 |
| Compressibility Index (%) | 22.727 | 18.293 |
| Hausner's Ratio | 1.294 | 1.224 |
| Tablet Core Parameters | | |
| Tablet Weight (mg) | 960-970 | 970-980 |
| Hardness (Newtons) | 90-100 | 90-100 |
| Thickness (mm) | 6.90-7.00 | 6.80-6.90 |
| Disintegration Time (min) | 0 min 24 sec | 0 min 50 sec |
| Friability (%) | 0.4 | 3.2 |

The desired hardness (above 120 N) could not be achieved without a sugar alcohol present in the granules.

We claim:

1. Granules comprising pirfenidone, a sugar alcohol, optionally a disintegrant, and optionally a pharmaceutical excipient, wherein the weight ratio of the sugar alcohol to the pirfenidone is 2:100 to 30:100.

2. The granules according to claim 1, wherein the granules do not contain a glidant.

3. The granules according to claim 1, wherein the pharmaceutical excipient is one of a binder and a filler.

4. The granules according to claim 3, wherein the granules further comprise a disintegrant.

5. The granules according to claim 1, wherein the sugar alcohol is a C₄ to C₁₂ sugar alcohol.

6. The granules according to claim 5, wherein the sugar alcohol is one of erythritol, xylitol, sorbitol, mannitol, maltitol, lactitol, and isomalt.

7. The granules according to claim 5, wherein the sugar alcohol is selected from the group consisting of mannitol or isomalt.

8. A tablet or capsule for oral administration containing the granules according to claim 1.

9. A process for preparing a capsule containing pirfenidone granules comprising the steps of:
(i) preparing a mixture comprising an amount of pirfenidone, a sugar alcohol, optionally a first disintegrant and optionally a further first pharmaceutical excipient;
(ii) subjecting the mixture obtained in step (i) to compaction;
(iii) milling the compacted material obtained in step (ii) to obtain the granules, wherein the weight ratio of the sugar alcohol to pirfenidone in the obtained granules is 2:100 to 30:100;
(iv) optionally mixing the granules with a second disintegrant or a further second pharmaceutical excipient; and
(v) filling the granules obtained in step (iii) or the mixture obtained in step (iv) into a capsule shell.

10. The process according to claim 9, wherein the step of compaction comprises roller compaction or slugging.

11. A process for preparing a capsule containing pirfenidone granules comprising the steps of:
(i) preparing a mixture consisting essentially of an amount of pirfenidone, a disintegrant, and a sugar alcohol;
(ii) subjecting the mixture obtained in step (i) to compaction;
(iii) milling the compacted material obtained in step (ii) to obtain the granules;
(iv) mixing the granules obtained in step (iii) with a lubricant; and
(v) filling the mixture obtained in step (iv) into a capsule.

12. The process according to claim 11, wherein the disintegrant is croscarmellose sodium, the sugar alcohol is isomalt and the lubricant is magnesium stearate.

13. A process for preparing a tablet from pirfenidone granules comprising the steps of:
(i) preparing a mixture comprising an amount of pirfenidone, a sugar alcohol, optionally a first disintegrant and optionally a further first pharmaceutical excipient;
(ii) subjecting the mixture obtained in step (i) to wet granulation using an aqueous granulating liquid to obtain the granules, wherein the weight ratio of the sugar alcohol to pirfenidone in the obtained granules is 2:100 to 30:100;
(iii) optionally mixing the granules obtained in step (ii) with a second disintegrant or a further second pharmaceutical excipient;
(iv) subjecting the granules obtained in step (ii) or the mixture obtained in step (iii) to compression to obtain the tablet; and
(v) optionally coating the tablet with a film.

14. The process according to claim 13, wherein the first pharmaceutical excipient is a filler, the aqueous granulating liquid contains a binder and the second pharmaceutical excipient is selected from a filler, a glidant and a lubricant.

15. A process for preparing a tablet from pirfenidone granules comprising the steps of:
(i) preparing a mixture consisting essentially of an amount of pirfenidone, a first disintegrant, a sugar alcohol and optionally a first filler;
(ii) subjecting the mixture obtained in step (i) to wet granulation using an aqueous binder-containing solution as granulating liquid to obtain the granules;
(iii) mixing the granules obtained in step (ii) with a second disintegrant, a second filler, a glidant and a lubricant;
(iv) subjecting the mixture obtained in step (iii) to compression to obtain the tablet; and
(v) optionally coating the tablet with a film.

16. The process according to claim 15, wherein the first and second disintegrant is croscarmellose sodium, the sugar alcohol is mannitol, the optionally contained first filler and the second filler are microcrystalline cellulose, the glidant is silicon dioxide and the lubricant is sodium stearyl fumarate.
